(19) United States
(12) Patent Application Publication
(10) Pub. No.: US 2013/0224754 A1
(43) Pub. Date: Aug. 29, 2013

(54) METHOD FOR THE SELECTION OF COMPOUNDS USEFUL FOR THE TREATMENT OF PSYCHIATRIC AND NEURODEGENERATIVE DISEASES

(71) Applicants: Enrico Tongiorgi, Trieste (IT); Valentina Vaghi, Villa Guardio (IT); Annalisa Vicario, Moruzzo (IT); Gabriele Baj, Varese (IT)

(72) Inventors: Enrico Tongiorgi, Trieste (IT); Valentina Vaghi, Villa Guardio (IT); Annalisa Vicario, Moruzzo (IT); Gabriele Baj, Varese (IT)

(73) Assignee: UNIVERSITA' DEGLI STUDI DI TRIESTE, Trieste (IT)

(21) Appl. No.: 13/884,130

(22) PCT Filed: Nov. 9, 2010
(86) PCT No.: PCT/EP2010/067081

§ 371 Date: May 8, 2013

(51) Int. Cl. G01N 33/50

(52) U.S. Cl. CPC G01N 33/5008
USPC 435/6.13; 435/320.1

(57) ABSTRACT

The present invention concerns a method for the identification of specific compounds which modulate brain-derived neurotrophic factor, which can be used as drugs for the treatment of neurological and neuropsychiatric diseases and which are suitable for the treatment of deleterious effects caused to the nervous system by abuse of illegal or legal drugs.

### FIELD OF THE INVENTION

The present invention concerns a method for the identification of specific compounds able to modulate Brain-derived neurotrophic factor (BDNF), which can be used as drugs for the treatment of neurological and neuropsychiatric diseases and which are suitable for the treatment of deleterious effects caused to the nervous system by abuse of illegal or legal drugs.

### STATE OF THE ART

Brain-derived neurotrophic factor (BDNF) is a growth and trophic factor that belongs to the family of related secretory proteins called neurotrophins. BDNF plays multifaceted and in part opposed functions in both development and maintenance of the nervous system. Indeed, BDNF promotes both cell survival and cell death, neuronal maturation including neurites and dendritic spine outgrowth, and has a prominent role in various forms of synaptic plasticity such as long-term potentiation and long-term depression (Lu, Hidaka et al. 1996; Huang and Reichardt 2001).

Transcription of the BDNF gene produces 11 primary transcripts in rodents (Aid, Kazantseva et al. 2007; Gene ID: 12064 and 24225) and 17 primary transcripts in humans (Pruunsild, Kazantseva et al. 2007; Gene ID: 627) each characterized by a different 5′ untranslated (UTR) exon linked by alternative splicing to a common 3′ exon coding for the protein and 3′UTR. Since the 3′UTR contains two polyadenylation sites, each primary transcript can exist in two forms, one with a short and the other with a long 3′ UTR, producing a total of 22 (in rodents) or 34 (in humans) possible transcripts.

Intraperitoneal injection of pro-convulsant agents such as kainic acid or pilocarpine in adult rats, induces a segregation of exon1 and 4 BDNF transcripts in the cell soma of hippocampal and cortical neurons, whereas exons 2B, 2C, and 6 transcripts display a somatodendritic distribution extended into distal dendrites (Pattabiraman, Tropea et al. 2005; Chiaruttini, Sonego et al. 2008). This differential local expression of BDNF transcripts appears to be a general mechanism used throughout the nervous system as it is found in cortex, hippocampus and hypothalamus (Pattabiraman, Tropea et al. 2005; Chiaruttini, Sonego et al. 2008; Aliaga, Mendoza et al. 2009). Of note, BDNF protein generated from each individual transcript segregates to the same subcellular domain where the transcript that has generated it localizes, regulating BDNF availability in specific subcellular regions of the neurons (Tongiorgi and Baj 2008). On this basis, it has been proposed that BDNF transcripts may represent a spatial code in which the different variants are used for the delivery of BDNF mRNA and accumulation of locally produced protein at specific subsets of synapses ((Chiaruttini, Sonego et al. 2008; Tongiorgi and Baj 2008).

Since pyramidal neurons of cortical layer 5 in the visual cortex as well as hippocampal neurons, receive segregated synaptic inputs from GABAergic interneurons on the cell soma and glutamatergic inputs on dendrites, the different BDNF transcripts may be recruited to modulate specifically different types of synaptic contacts (Tongiorgi, 2008). Accordingly, mice with a selective ablation of the “somatic” exon 4 transcript were shown to exhibit significant deficits in GABAergic interneurons in the prefrontal cortex, particularly those expressing parvalbumin, a subtype implicated in executive function and schizophrenia. Moreover, disruption of promoter 4-driven BDNF transcription impaired inhibitory but not excitatory synaptic transmission recorded from layer 5 pyramidal neurons in the prefrontal cortex (Sakata, Woo et al. 2009).

Numerous studies have reported changes in the expression of the different BDNF mRNA variants in various brain diseases such as epilepsy (Aid, Kazantseva et al. 2007) and mental retardation Rett Syndrome (Chen, Chang et al. 2003), or neurodegenerative diseases like Huntington disease (Zuccato, Liber et al. 2005) and Alzheimer's disease (Garzon, Yu et al. 2002). Moreover, BDNF variants levels are different from normal brains in various human brain regions from cocaine addicts. The cocaine group showed threefold higher levels of exon 4-specific mRNAs in cerebellum versus controls and a 40% reduction of exon 4 and exon 1-specific BDNF mRNA in the cortex (Jiang, Zhou et al. 2009). Fear and stress can also specifically affect BDNF transcripts expression levels. A recent study reported evidence of an epigenetic regulation of BDNF gene transcription in the consolidation of fear memory. Contextual fear learning induced differential regulation of exon-specific BDNF mRNAs (1, 4, 6, 9a) that was associated with changes in BDNF DNA methylation and altered local chromatin structure (Roth, Lubin et al. 2009).

Pruunsild et al. (2007), and Aid et al. (2007) studies have highlighted that the 5′UTR region of each BDNF transcript is encoded by a different exon with unique length, GC content and putative secondary structures. Thus, each BDNF transcript is likely to display a different translatability. However, since the final protein product is the same, it is presently impossible to determine the relative contribution of each single BDNF splice variants to the production of the BDNF protein. Thus, to determine the role of each BDNF transcript in producing the protein, it is necessary to develop new tools able to determine the amount of protein generated by each BDNF splice variant at basal condition and in response to a disease state or a specific drug. Clearly, a change in mRNA levels of transcript that is poorly translatable will have less impact on total BDNF levels than a highly translatable splice variant.

The need and importance is increasingly felt for the identification of specific compounds able to modulate BDNF, which can be used as drugs for the treatment of neurological and neuropsychiatric diseases or for the treatment of deleterious effects caused to the nervous system by abuse of illegal or legal drugs. It is therefore object of the present invention the development of a method of screening for BDNF translation modulators which allow the measurement of BDNF protein production. In particular it would be highly desirable to identify a screening method which would allow to determine the expression of all possible BDNF variants, and allow, at the same time, to obtain information on the final amounts of the BDNF protein produced in response to a specific drug.

### SUMMARY OF THE INVENTION

Object of this invention is the creation of a cell-based screening assay to screen for natural or synthetic compounds able to increase or decrease the neurotrophin brain-derived neurotrophic factor (BDNF) protein levels produced by translation of the different BDNF mRNA variants.

In particular, the present invention concerns a method of screening for modulators of the translation of brain-derived neurotrophic factor (BDNF) comprising the steps of:

a) transfecting a mammalian cell with a nucleic acid construct, said nucleic acid construct comprising:
a reporter gene; and
a 5′ untranslated (5′UTR) exon of a mammalian BDNF gene

b) contacting said mammalian cell with a screening compound;

c) detecting the luminescence produced by said mammalian cell of step b).

A further aspect of the present invention is a method of screening for modulators of the translation of a brain-derived neurotrophic factor (BDNF) comprising the steps of:

a) transfecting a mammalian cell with a nucleic acid construct, said nucleic acid construct comprising:
a reporter gene; and
a 3′ untranslated (3′UTR) exon of a mammalian BDNF gene;

b) contacting said mammalian cell with a screening compound;

c) detecting the luminescence produced by said mammalian cell of step b).

The present invention further concerns a nucleic acid construct comprising either:
a) a reporter gene; and
b) a 5′ untranslated (5′UTR) exon of the rat BDNF gene selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 or SEQ ID NO. 10;
or
a) a reporter gene; and
b) a 3′ untranslated (3′UTR) exon of the rat BDNF gene selected from the group consisting of SEQ ID NO. 13 and SEQ ID NO. 14;
or
a) a nucleic acid construct comprising a 5′ untranslated (5′UTR) exon of the rat BDNF gene selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 or SEQ ID NO. 10, and a reporter gene followed by either the short or the long version of the 3′UTR of the rat BDNF gene selected from the group consisting of SEQ ID NO. 13 and SEQ ID NO. 14.

A still further object of the present invention is a plasmid comprising the nucleic acid construct according to the present invention.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-6, wherein:

FIG. 1: shows the strategy of cloning for creation of the new pFluc-N1 and pRluc vectors described in Example 1.

FIG. 2A: shows the mRNA levels for each transcript transfected in SH-SY5Y and PCR-amplified with primers specific for the luciferase reporter gene as described in Example 2;

FIG. 2B: shows the densitometric analysis normalized for the levels of the housekeeping gene GAPDH revealed some variability in mRNA expression for the different variants as described in Example 2;

FIG. 2C: shows the results of normalization of the luciferase translation data for the levels of the corresponding mRNA lead to the results as described in Example 3;

FIG. 2D shows the results of a western-blot, showing the protein expression (antibody anti-luc) for each pN1-insert name as described in Example 3.

FIG. 3: shows the effects of drug treatment on the translation of BDNF 5′UTRs as described in Example 4.

FIG. 4: shows MTT viability assays demonstrating that none of the drugs used at the indicated concentrations causes death in SY-SY5Y human neuroblastoma cells as described in Example 4.

FIG. 5: shows the effects of drug treatment on the translation of BDNF 3′UTRs as described in Example 5.

FIG. 6: shows the inhibition of KCl-induced translation of the ex6-Fluc-3′UTRlong construct as described in Example 6.

### DETAILED DESCRIPTION OF THE INVENTION

The present invention concerns a method of screening for modulators of the translation of brain-derived neurotrophic factor (BDNF) comprising the steps of:

a) transfecting a mammalian cell with a nucleic acid construct, said nucleic acid construct comprising:
a reporter gene; and
a 5′ untranslated (5′UTR) exon of a mammalian BDNF gene;

b) contacting said mammalian cell with a screening compound;

c) detecting the luminescence produced by said mammalian cell of step b).

A further object of the present invention is a method of screening for modulators of brain-derived neurotrophic factor (BDNF) translation, wherein said nucleic acid construct further comprises a 3′ untranslated (3′UTR) exon of a mammalian BDNF gene;

A further aspect of the present invention is a method of screening for modulators of the translation of a brain-derived neurotrophic factor (BDNF) comprising the steps of:

a) transfecting a mammalian cell with a nucleic acid construct, said nucleic acid construct comprising:
a reporter gene; and
a 3′ untranslated (3′UTR) exon of a mammalian BDNF gene;

b) contacting said mammalian cell with a screening compound;

c) detecting the luminescence produced by said mammalian cell of step b).

In a preferred aspect, the present invention regards a method of screening for modulators of the translation of brain-derived neurotrophic factor (BDNF), wherein said 5′ untranslated (5′UTR) exon of a mammalian BDNF gene is the 5′ untranslated (5′UTR) exon of the rat BDNF gene.

In a more preferred embodiment, the present invention regards a method of screening for modulators of the translation of brain-derived neurotrophic factor (BDNF), wherein said 5′ untranslated (5′UTR) exon of the rat BDNF gene is selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 or SEQ ID NO. 10.

For the purposes of the present invention, each 5′ untranslated (5′UTR) exon of the rat BDNF gene has a corresponding SEQ ID NO. as follows:

SEQ ID NO. 1 corresponds to the nucleotidic sequence of exon 1 of the 5′ untranslated (5′UTR) exon of the rat BDNF gene;

SEQ ID NO. 2 corresponds to the nucleotidic sequence of exon 2a of the 5′ untranslated (5′UTR) exon of the rat BDNF gene;

SEQ ID NO. 3 corresponds to the nucleotidic sequence of exon 2b of the 5′ untranslated (5′UTR) exon of the rat BDNF gene;

SEQ ID NO. 4 corresponds to the nucleotidic sequence of exon 2c of the 5′ untranslated (5′UTR) exon of the rat BDNF gene;

SEQ ID NO. 5 corresponds to the nucleotidic sequence of exon 3 of the 5′ untranslated (5′UTR) exon of the rat BDNF gene;

SEQ ID NO. 6 corresponds to the nucleotidic sequence of exon 4 of the 5′ untranslated (5′UTR) exon of the rat BDNF gene;

SEQ ID NO. 7 corresponds to the nucleotidic sequence of exon 5 of the 5′ untranslated (5′UTR) exon of the rat BDNF gene;

SEQ ID NO. 8 corresponds to the nucleotidic sequence of exon 6 of the 5′ untranslated (5′UTR) exon of the rat BDNF gene;

SEQ ID NO. 9 corresponds to the nucleotidic sequence of exon 7 of the 5′ untranslated (5′UTR) exon of the rat BDNF gene;

SEQ ID NO. 10 corresponds to the nucleotidic sequence of exon 8 of the 5′ untranslated (5′UTR) exon of the rat BDNF gene.

For the purposes of the present invention, by the term “BDNF”, it is intended any gene of a mammalian brain-derived neurotrophic factor which can generate any isoform of the BDNF protein such as proBDNF, truncated or mature BDNF, in particular a rat BDNF. In addition to rat BDNF, BDNF sequences for human and other mammalian species are known in the art and sequences of BDNF from these other species can be found on the NCBI website and in other resources known to persons of skill in the art.

According to a preferred aspect, the present invention regards a method of screening for modulators of the translation of brain-derived neurotrophic factor (BDNF), wherein said 3′ untranslated (3′UTR) exon of a mammalian BDNF is the 3′ untranslated (3′UTR) exon of rat BDNF gene.

In a more preferred embodiment, the present invention regards a method of screening for modulators of the translation of brain-derived neurotrophic factor (BDNF), wherein said 3′ untranslated (3′UTR) exon of the rat BDNF gene is selected from the group consisting of SEQ ID NO. 13 and SEQ ID NO. 14.

For the purposes of the present invention, each 3′ untranslated (3′UTR) exon of the rat BDNF gene has a corresponding SEQ ID NO. as follows:

SEQ ID NO. 13 corresponds to the nucleotidic sequence of the short 3′ untranslated region of the rat BDNF gene.

SEQ ID NO. 14 corresponds to the nucleotidic sequence of the long 3′ untranslated region of the rat BDNF gene.

In the present invention by “screening compound”, a compound that could be a promising candidate for drug development is intended. Such chemical compounds interact with a target protein and are therefore potential candidates for drug development. The target protein of the present invention is a protein involved in the signaling cascades which regulate the translation of a transcript encoding BDNF and of which non-limiting examples are represented by: 1) ion channels; 2) protein involved in neurotransmitters, peptides, hormons, and trophic factors release, synthesis or re-uptake; 3) receptors for neurotransmitters, peptides, hormons and trophic factors; 4) enzymes involved in post-translational modifications of proteins of which non-limiting examples are represented by phosphorylation, acetylation, or sumoylation; 5) enzymes involved in production of small signaling molecules of which non-limiting examples are represented by nitric-oxide, cyclic nucleotides, phospholipids; 6) translation factors or proteins regulating the activity of translation factors, and 7) RNA-binding proteins or proteins regulating the activity of RNA-binding proteins involved in RNA synthesis, trafficking, folding, translation or degradation.

The method of the present invention has the advantage of being useful for the identification of specific compounds able to modulate BDNF, which can be used as drugs for the treatment of neurological and neuropsychiatric diseases or for the treatment of deleterious effects caused to the nervous system by abuse of illegal or legal drugs.

A further advantage of the method of the present invention is that of allowing the screening for BDNF translation modulators which allow the measurement of BDNF protein production, and in particular allows to determine the expression of all possible BDNF variants, and allow, at the same time, to obtain information on the final amounts of the BDNF protein produced in response to a specific drug.

A further object of the present invention is a method of screening for modulators of brain-derived neurotrophic factor (BDNF), wherein said mammalian cell grows in adhesion.

In the present invention, said mammalian cell is chosen from the group consisting of human neuroblastoma-derived cell lines of which non-limiting examples are represented by SH-SY5Y, SK-N-SH, and SK-N-BE cells, or glioblastoma-derived cell lines of which non-limiting examples are represented by the GBM cell lines U87, U343, U563 and the MB cell lines D283, DAOY, and SNB40 or immortalized human cell lines of neuronal origin of which non-limiting examples are represented by human neural progenitor cells (hNPCs), human embryonic spinal cord-derived cell line (HSP1), human dorsal root ganglion cell line, human cortical neural progenitor cells, or of human cell lines of non-neuronal origin of which non-limiting examples are represented by NIH-3T3 cells, HeLa cells, or rodent immortalized cell lines of which non-limiting examples are represented by HEK 293 cells, and CHO cells.

In a preferred embodiment, the present invention concerns a method of screening for modulators of BDNF, wherein said mammalian cell is chosen from the group consisting of SH-SY5Y cells, SK-N-SH cells, SK-N-BE cells, NIH-3T3 cells, HeLa cells, HEK 293 cells, and CHO cells.

The present invention further relates to a method of screening for modulators of BDNF, wherein said screening compound is a BDNF-translation agonist.

The present invention further relates to a method of screening for modulators of BDNF, wherein said screening compound is selected from the group consisting of ion-channels agonists, neurotransmitters synthesis, release and re-uptake agonists, neurotransmitter receptors agonists, growth-factors receptors agonists, translation factors agonists and intracellular signaling agonists.

The present invention further relates to a method of screening for modulators of BDNF, wherein said screening compound is a BDNF-translation antagonist.

The present invention further relates to a method of screening for modulators of BDNF, wherein said screening compound is selected from the group consisting of ion-channels antagonists, neurotransmitters synthesis, release and re-uptake antagonists, neurotransmitter receptors antagonists, growth-factors receptors antagonists, translation factors antagonists and intracellular signaling antagonists.

Object of this invention is therefore the creation of a cell-based screening assay to screen for natural or synthetic compounds able to treat neurological diseases through an increase or decrease of BDNF protein levels produced by translation of the different BDNF splice variants.

In a further embodiment, the present invention regards a method of screening for modulators of BDNF, wherein said reporter gene is a luciferase gene.

In a further embodiment, the present invention regards a method of screening for modulators of BDNF, wherein said luciferase gene is chosen from the group consisting of Firefly luciferase and Renilla luciferase.

For the purposes of the present invention, each luciferase gene has a corresponding SEQ ID NO. as follows:

SEQ ID NO. 11 corresponds to the nucleotidic sequence of the luciferase gene of the firefly Photinus pyralis (full coding sequence)

SEQ ID NO. 12 corresponds to the nucleotidic sequence of the luciferase gene of the sea pansy Renilla reniformis (full coding sequence).

In a still further embodiment the present invention regards a method of screening for modulators of the translation of a brain-derived neurotrophic factor (BDNF), wherein said method of screening is carried out by the high throughput screening (HTS) technique.

In the present invention by the High-throughput screening (HTS) technique, the technique which seeks to identify potential candidates for drug development is intended. HTS is a technique for scientific experimentation especially used in drug discovery. Using robotics, data processing and control software, liquid handling devices, and sensitive detectors, HTS allows to quickly conduct millions of biochemical, genetic or pharmacological tests.

In a still further embodiment the present invention regards a nucleic acid construct comprising either:

a) a reporter gene; and

b) a 5′ untranslated (5′UTR) exon of the rat BDNF gene selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 or SEQ ID NO. 10;

or

a) a reporter gene; and

b) a 3′ untranslated (3′UTR) exon of the rat BDNF gene selected from the group consisting of SEQ ID NO. 13 and SEQ ID NO. 14.

or

a) a reporter gene; and

b) a 5′ untranslated (5′UTR) exon of the rat BDNF gene selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 or SEQ ID NO. 10; and

c) a 3′ untranslated (3′UTR) exon of the rat BDNF gene selected from the group consisting of SEQ ID NO. 13 and SEQ ID NO. 14.

In a still further embodiment the present invention regards a nucleic acid construct, wherein said reporter gene is a luciferase gene.

In a still further embodiment the present invention regards a nucleic acid construct, wherein said luciferase gene is chosen from the group consisting of Firefly luciferase and Renilla luciferase.

In a still further embodiment the present invention regards a plasmid comprising the nucleic acid construct according to the present invention.

### EXAMPLES

### Example 1

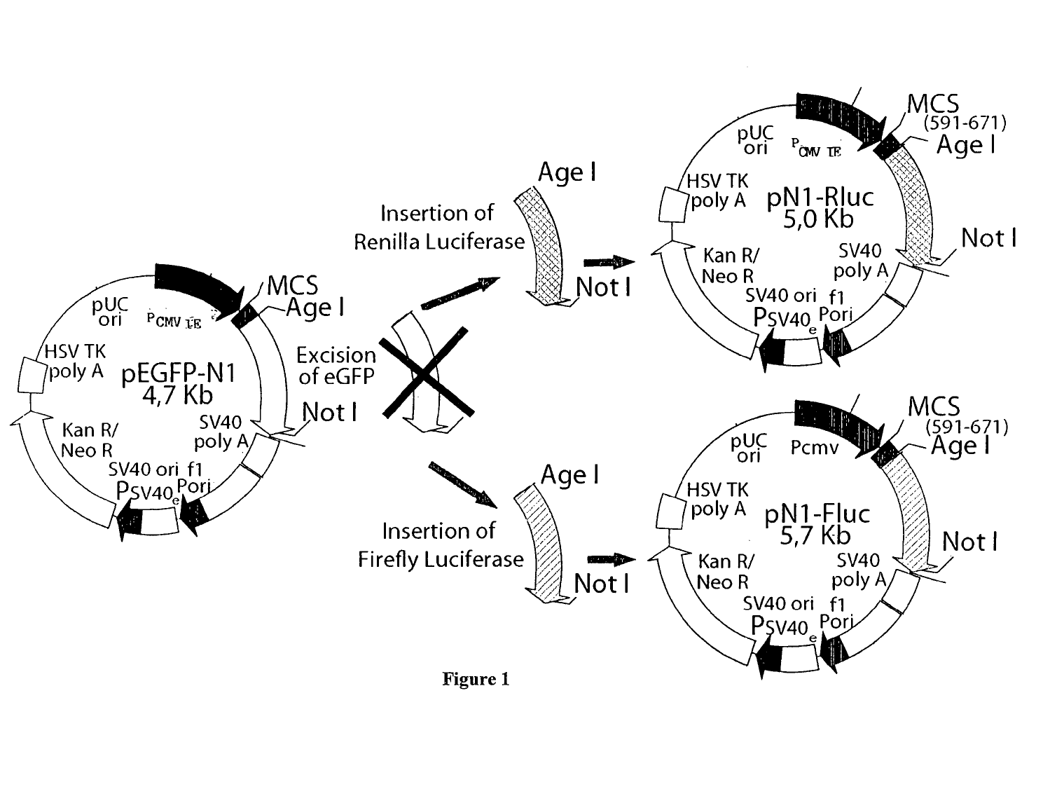

Cloning and Generation of Luciferase Constructs pN1-RLuc and pN1-FLuc as Shown in FIG. 1

Two different constructs were generated by modifying the commercial pEGFP-N1 plasmid (Clontech). pN1-Rluc was obtained by the replacement of the EGFP coding sequence of pEGFP-N1, with the Renilla-luciferase (GI:2582516; GB:AAB82577.1). pN1-FLuc was obtained by the replacement of the EGFP coding sequence of pEGFP-N1, with the Firefly-luciferase open reading frame (ORF) (GI:13195704; GB:AAA89084.1).

The cloning strategy and the resulting maps of the newly obtained pN1-FLuc (pFluc) and pN1-RLuc (or pRluc) are shown in FIG. 1. pN1-FLuc (pFluc) and pN1-RLuc (or pRluc) were then used as a backbone to construct the vectors containing the different BDNF splicing variants.

Cloning Strategy

In detail, the pEGFP-N1 vector was modified so that the sequence between the restriction sites Age I and Not I, encoding the EGFP (enhanced green fluorescent protein) was removed. The digested backbone vector was purified through DNA agarose gel extraction (Sigma Aldrich, Gel Extraction kit). The Firefly luciferase DNA fragment (Rluc) was obtained by PCR amplification of the luc+ gene from the commercial vector pGL3 Basic and the Renilla luciferase DNA fragment (Rluc) was obtained by PCR amplification of the Rluc gene from the pRL-SV40 Vector (both purchased from Promega Corporation). The Fluc and Rluc ORFs were amplified using Phusion high-fidelity DNA Polymerase (Finnzymes) 0.02 U/μl. Primers were purchased from Eurofins MWG GmbH. The PCR conditions and primers sequence are summarized in the Table 1.

TABLE 1
PCR primers and conditions for amplifying Rluc and Fluc

| | Primer Forward | Primer Reverse | PCR CONDITIONS | | | |
|---|---|---|---|---|---|---|
| Fluc | GCCACCGGTCGCCACCATGGA | CGGGCGGCCGCTTTACACGGC | Denatur- | 98° C. | 10 sec | 30 cycles |
| | AGACGCC | GATCTTTCCGC | ation | | | |
| | | | Annealing | 58° C. | 20 sec | |
| | | | Extension | 72° C. | 40 sec | |
| Rluc | GCCACCGGTCGCCACCATGAC | CGGGCGGCCGCTGGGCCCGTT | Denatur- | 98° C. | 10 sec | 30 cycles |
| | TTCGAAAGTTTATGATCC | GTTCATTTTTGAGAACTCG | ation | | | |
| | | | Annealing | 57.5° C. | 20 sec | |
| | | | Extension | 72° C. | 30 sec | |

PCR-amplified Fluc and Rluc fragments were cut with the restriction enzymes AgeI and NotI (New England Biolabs) and then ligated into the pEGFP-N1 vector backbone without EGFP (as described above).

In order to obtain one specific expression vector for each BDNF variant (from now on, named pN1-insert name-Fluc), the sequence of single rat BDNF exons 1-8 encoding the alternatively spliced 5′UTR regions, was amplified by PCR starting from the cDNA obtained from total RNA extracted from adult rat brain and retro-transcribed using Superscript-II transcriptase (Promega). The forward primers, specific for each 5′exon contained the XhoI restriction site and the common reverse primer in the coding region contained the AgeI restriction site (Table 2). PCR amplicons were cloned between the XhoI and AgeI restriction sites of the pFluc-N1 vector upstream of the Firefly Luciferase coding sequence. In addition, the complete rat BDNF coding sequence (CDS) was cloned between the XhoI and SacII restriction site of the pFluc-N1 vector upstream the Fluc gene.

Furthermore, the two 3′untranslated regions (3′UTR) variants, called respectively 3′UTR-long and 3′UTR-short were cloned into the HpaI and NotI restriction sites of pN-Fluc vector downstream the F-luc gene using specific primers generating specific vectors (from now on named, pN1-Fluc-3′UTR-long or short; or pN1-insert name-Fluc-3′UTR-long or short. See Table I). Finally, the two Kozak sequences, one specific for exon 1 and the other in common to all other transcripts (exon 9), were inserted (AgeI-NotI) through a modified Fluc sequence containing the two above modified kozak sequences. The BDNF CDS, 3′UTR-long and 3′UTR-short and all exons were amplified using Phusion high-fidelity DNA Polymerase (Finnzymes) 0.02 U/μl (or Platinum Polymerase for exon 3). Primers were purchased from Eurofins MWG GmbH (see Table 2 for PCR conditions).

Results

The plasmids generated with the cloning strategy described above are listed in Table 3.

TABLE 2
PCR primers and conditions for pN1-BDNF insert -Fluc vectors

| | Primer Forward | Primer Reverse | PCR CONDITIONS | | | |
|---|---|---|---|---|---|---|
| 3′ | GGGCGGCCGCTGGATTTATGTTGTA | GCCGGTTAACTTACAATAGGCTTCTG | Denatur- | 98° C. | 10 sec | 31 |
| UTR | TAG | ATGTGG | ation | | | cycles |
| long | | | Annealing | 57.5° C. | 15 sec | |
| | | | Extension | 72° C. | 2 min | |
| 3′ | GGGCGGCCGCTGGATTTATGTTGTA | GTCAGTTAACTTTATTATCAATTCACA | Denatur- | 98° C. | 10 sec | 31 |
| UTR | TAG | ATTAAAGC | ation | | | cycles |
| short | | | Annealing | 57.5° C. | 15 sec | |
| | | | Extension | 72° C. | 15 sec | |
| CDS | GCGCTCGAGATGACCATCCTTTTCCT | GTCACACGTGTCCCCTTTTAATGGTC | Denatur- | 98° C. | 10 sec | 31 |
| | TAC | AGTG | ation | | | cycles |
| | | | Annealing | 57.5° C. | 15 sec | |
| | | | Extension | 72° C. | 40 sec | |
| Exon | AATTCTCGAGTAAAGCGGTAGCCGG | GTCTACCGGTTTTGCTGTCCTGGAGA | Denatur- | 98° C. | 10 sec | 31 |
| 1 | CTGGTGCAGG | CTCAGTGTC | ation | | | cycles |
| | | | Annealing | 57° C. | 20 sec | |
| | | | Extension | 72° C. | 40 sec | |
| Exon | GATCCTCGAGGCTTTGGCAAAGCCA | GCCACCGGTCTGGATGAAGTACTAC | Denatur- | 98° C. | 10 sec | 31 |
| 2A | TCCGCACGTGAC | CACCTCGGAC | ation | | | cycles |
| | | | Annealing | 58° C. | 20 sec | |
| | | | Extension | 72° C. | 35 sec | |
| Exon | GATCCTCGAGGCTTTGGCAAAGCCA | GTTACCGGTGGAGCTTGCCAAGAGT | Denatur- | 98° C. | 10 sec | 31 |
| 2B | TCCGCACGTGAC | CTATTCCAG | ation | | | cycles |
| | | | Annealing | 58° C. | 20 sec | |
| | | | Extension | 72° C. | 35 sec | |
| Exon | GATCCTCGAGGCTTTGGCAAAGCCA | GAAACCGGTCTTCTTTGCGGCTTACA | Denatur- | 98° C. | 10 sec | 31 |
| 2C | TCCGCACGTGAC | CCACCCCGGTGGCTAG | ation | | | cycles |
| | | | Annealing | 58° C. | 20 sec | |
| | | | Extension | 72° C. | 35 sec | |
| Exon | AGGCTCGAGGCCCTCACGATTCTCGC | GCTAACCGGTCTGGGCTCAATGAAG | Denatur- | 94° C. | 15 sec | 31 |
| 3* | TGGATAG | CATCCAGCCC | ation | | | cycles |
| | | | Annealing | 55° C. | 15 sec | |
| | | | Extension | 68° C. | 20 sec | |
| Exon | AATTCTCGAGACCCACTTTCCCATTC | GACTACCGGTCAGTCACTACTTGTCA | Denatur- | 98° C. | 10 sec | 31 |
| 4 | ACCGAGG | AAGTAAACATCAAGGC | ation | | | cycles |
| | | | Annealing | 58° C. | 20 sec | |
| | | | Extension | 72° C. | 20 sec | |
| Exon | GACTCTCGAGAAACCATAACCCCGCA | GACTACCGGTCTTCCCGCACCTTCCC | Denatur- | 98° C. | 10 sec | 31 |
| 5 | CACTCTGTGTAGTTTCATTGTGTGTTC | GCACCACAGAGCTAGAAAAAGCGAA | ation | | | cycles |
| | G | CACAC | Annealing | 58° C. | 20 sec | |
| | | | Extension | 72° C. | 15 sec | |
| Exon | GACTCTCGAGCCAATCGAAGCTCAAC | GATCACCGGTCTCAGGGTCCACACA | Denatur- | 98° C. | 10 sec | 31 |
| 6** | CGAAGAGC | AAGCTCTCGG | ation | | | cycles |
| | | | Annealing | 57.5° C. | 20 sec | |
| | | | Extension | 72° C. | 15 sec | |
| Exon | GACTCTCGAGCACTGTCACCTGCTTT | GTACACCGGTCTCCCGGATGAAAGT | Denatur- | 98° C. | 10 sec | 31 |
| 7 | CTAGGGAGTATTACC | CAAAACTTTCACTTCCTCTGGAGG | ation | | | cycles |
| | | | Annealing | 58° C. | 20 sec | |
| | | | Extension | 72° C. | 20 sec | |
| Exon | GTCACTCGAGGTTATAGAGTTGGAT | GCATACCGGTGACACCATTTTCAGCA | Denatur- | 98° C. | 10 sec | 31 |
| 8 | GCAAGCGTAACCCG | ATCGTTTGTTCAGCTCC | ation | | | cycles |
| | | | Annealing | 58° C. | 20 sec | |
| | | | Extension | 72° C. | 35 sec | |

Complete list of the plasmids

| | |
|---|---|
| 1) | pN1-Rluc |
| 2) | pN1-Fluc |
| 3) | pN1-Fluc-3′short |
| 4) | pN1-Fluc-3′long |
| 5) | pN1-CDS-Fluc |
| 6) | pN1-kozakex9-Fluc |
| 7) | pN1-exon2B-Fluc |
| 8) | pN1-exon2A-Fluc |
| 9) | pN1-exon8-Fluc |
| 10) | pN1-exon2C-Fluc |
| 11) | pN1-exon1-Fluc |
| 12) | pN1-exon4-Fluc |
| 13) | pN1-exon7-Fluc |
| 14) | pN1-exon5-Fluc |
| 15) | pN1-exon3-Fluc |
| 16) | pN1-exon6-Fluc |
| 17) | pNI-exon1-Fluc-3′UTR short |
| 18) | pNI-exon1-Fluc-3′UTR long |
| 19) | pN1-exon4-Fluc-3′UTR short |
| 20) | pN1-exon4-Fluc-3′UTR long |
| 21) | pN1-exon6-Fluc-3′UTR short |
| 22) | pN1-exon6-Fluc-3′UTR long |

medium was replaced with fresh medium after 24 h, before transfection.

Transfection

Plasmids were transfected into SH-SY5Y cells using lipofectamine 2000 (Invitrogen) to obtain a transient transfection. The relative quantity of DNA to lipofectamine has been determined and the optimal ratio was found at 0.2 μg of DNA and 0.5 μl of lipofectamine for each well of a 96 multiwell plate. At the time of transfection SH-SY5Y cells were around 70-75% of confluency. According to the Promega protocol for luciferase activity measurements, each construct-containing Fluc was transfected together with the normalizing Rluc construct with a 10:1 ratio (0.2 μg Fluc containing vector together with 0.02 μg of pN1-Rluc vector). The DNA (0.2 μg) was mixed with 25 μl of native MEM medium and incubated for 5 minutes at room temperature. The two solutions were then mixed and incubated for 20 minutes at room temperature to allow the formation of DNA-containing micelles. Then, 50 μl of such solution were added in each well to cells and incubated for 24 h at 37° C. After 24 h transfection, the medium was removed and replaced with fresh culture medium.

Results

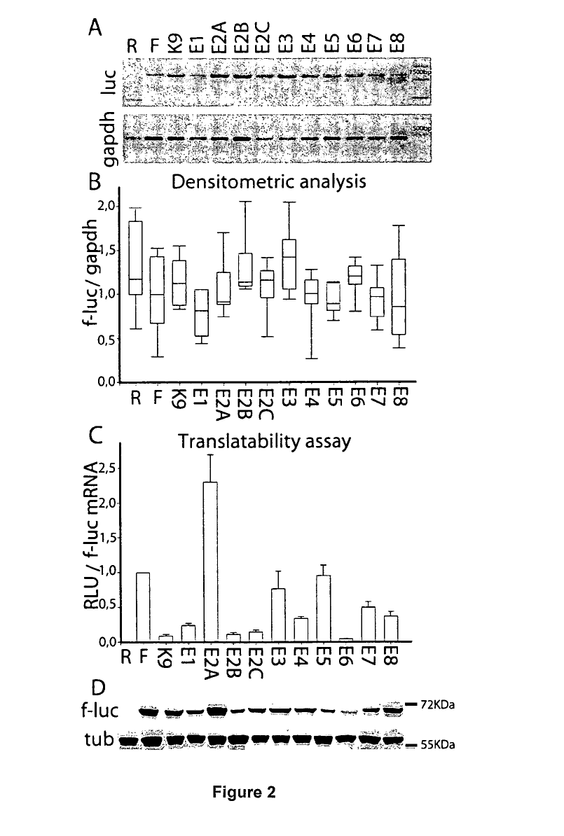

FIG. 2A shows the mRNA levels for each transcript transfected in SH-SY5Y and PCR-amplified with primers specific for the luciferase reporter gene. In this way each transcript is amplified with the same efficiency. Densitometric analysis (FIG. 2B) of three independent experiments (each in duplicate), normalized for the levels of the housekeeping gene GAPDH revealed some variability in mRNA expression for the different variants which was non-statistically significant.

### Example 3

Dual Luciferase Assay

Each vector was tested in the human neuroblastoma cell line SH-SY5Y and the basal levels of translation measured as F/R-luc ratio, were determined in three independent experiments (each in duplicate) in 96 well white plates with clear bottom (FBI International). To determine the effects of receptors activation on translation controlled by the different BDNF 5′UTR sequences, cells were treated for 3 hours with 50 mM KCl to induce generalized cell depolarization.

Firefly Luciferase Assay

Luciferase assay was performed according to the manufacturer's instructions (Promega). At the time of luciferase assay, the growth medium was removed from the cultured cells and a sufficient volume of PBS 1× was applied to wash the surface of the culture vessel. Then, the PBS 1× solution was completely removed and the cells lysis was started by adding 20 μl of PLB (passive lysis buffer) into each well. After adding the PLB solution, the culture plate was placed on an orbital shaker with gentle shaking at room temperature for 15 minutes to cover uniformly the cell monolayer with PLB. After lysis, 100 μl of Luciferase Assay Reagent II (LARII) were dispensed in each well and Firefly luciferase activity was measured using the Glo Max Multi luminometer with two injectors (Promega Corporation).

Renilla Luciferase Assay

After quantifying the Firefly luminescence, this reaction was quenched and simultaneously, the Renilla luciferase reaction was initiated by adding 100 μl Stop & Glo Reagent (Promega) to the same well. Then, Renilla luciferase activity was measured using the Glo Max Multi luminometer with two injectors (Promega Corporation).

Western-Blot Analysis

Adherent SH-SY5Y cells were mechanically removed with a cell scraper (Sarstedt) with 150 μl of cold (4° C.) lysis buffer containing 137 mM NaCl (Fluka), 20 mM Tris-HCl pH 8.0, 1% Nonidet P-40, 10% glycerol, and a cocktail of protease inhibitors—1 mM PMSF (Phenilmethyl-sulfonilfluoride), 10 μg/ml TEWI (Turkey Egg White inhibitor), 4 μg/ml SBTI (Soy Bean Trypsin inhibitor), 1 mM IAA (Iodoacetamide), 1 mM SPERM (Spermidin). SH-SY5Y extracts were further homogenized with a syringe, rocked for 30-45 min at 4° C., and centrifuged at 10,000×g for 8-10 min at 10° C., to remove cellular debris. Protein homogenates (30 μg) were separated on 12% SDS-polyacrylamide gels and transferred onto nitrocellulose Protran membranes (Whatman). After blocking (4% non-fat milk powder, 0.05% tween-20 in phosphate-buffered saline) at RT, membranes were divided into two halves at the level of the 60 KDa marker and incubated overnight at 4° C. with either a goat anti-firefly luciferase antibodies (AbCam; diluted 1:1000) or mouse monoclonal anti-tubulin antibody (YOL1/34, AbCam; diluted 1:2000). Following incubation with anti-goat or anti-mouse secondary antibodies conjugated with HRP (both DakoCytomation, diluted 1:10,000), immunoreactivity was detected by chemiluminescence (Amersham Biosciences). Films were scanned using an Epson Scanner (Epson perfection V500-photo).

Results

The different 5′UTR BDNF sequences have a different translatability. Normalization of the luciferase translation data for the levels of the corresponding mRNA lead to the results shown in FIG. 2C. Insertion of the different rat BDNF 5′UTR upstream to the luciferase gene resulted in a marked repression of translation of the reporter for most transcripts with the exception of exon 2A which enhanced translation. In FIG. 2D a representative Western-blot is displayed, showing the protein expression (antibody anti-luc) for each pN1-insert name-Fluc construct. Results of the luciferase assay and the western-blot are consistent with each other.

### Example 4

Response of BDNF 5′UTR Splice Variants to Different Agonists

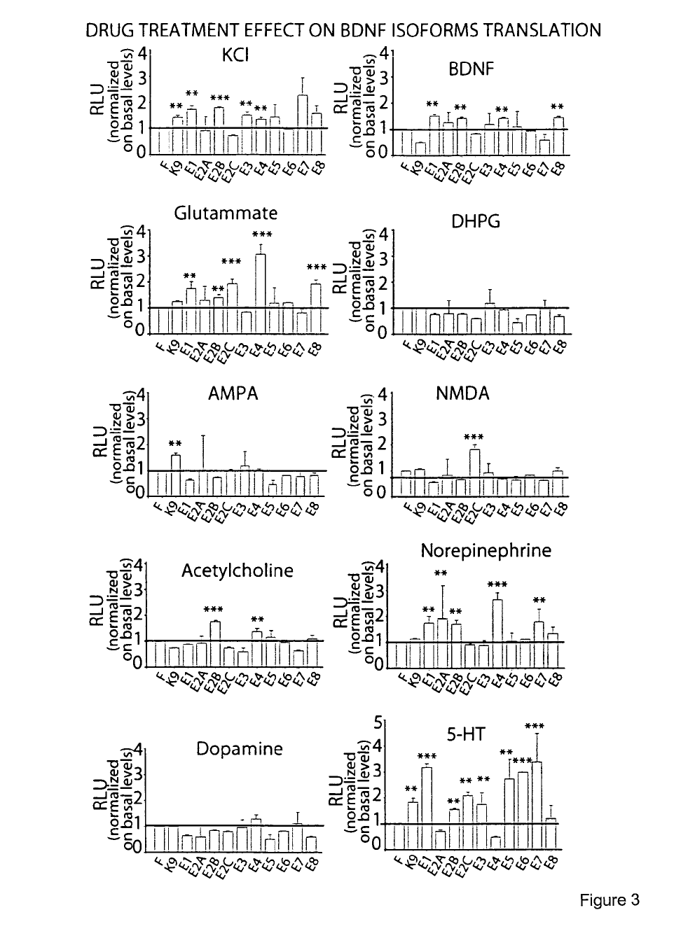

The different BDNF splice variants were analyzed for their response to different receptor agonists (FIG. 3) in undifferentiated neuro-blastoma SH-SY5Y cells. One day prior transfection, undifferentiated neuro-blastoma SH-SY5Y cells were seeded into a 96-well white plate with clear bottom (PBI International) at a confluency of 50-60% and cultured overnight. Plasmids containing the different BDNF 5′UTR exons were then transiently transfected into SH-SY5Y cells using Lipofectamine™ 2000 (Invitrogen) at high cell density (70-75%), according to the manufacturer's protocol. 24 hours after transfection, the cells were treated with the listed receptor agonists at the following concentrations: KCL (50 mM), BDNF (50 ng/ml), Glutammate (GLU: 20 μM), S)-3,5-Dihydroxyphenylglycine (DHPG: 50 μM), AMPA (30 μM), NMDA (30 μM), Acetylcholine (AcH: 30 μM), Norepinephrine (NE: 50 μM), Dopamine (DOPA: 40 μM) and Serotonin (5-HT 50 μM). All agonists were purchased from Sigma Aldrich with the exception of DHPG, AMPA and NMDA that were from Ascent Scientific. Then dual luciferase assay was performed as described according to the manufacturer's instructions (Promega).

MTT Viability Assay

Each drug was also tested in a viability assay to demonstrate that the concentration used was not toxic for the cells as no appreciable cell death could be detected. Using a 96-well plate 100 μl of SHSY-5Y cells were distributed per well, maintaining them overnight at 37° C. in a 5% CO₂-humified incubator. On the following day the cells were incubated for 3 h at 37° C. with the following drugs: KCl (50 mM), BDNF (50 ng/ml), Glutammate (GLU: 20 μM), S)-3,5-Dihydroxyphenylglycine (DHPG: 50 μM), AMPA (30 μM), NMDA (30 μM), Acetylcholine (AcH: 10 μM), Norepinephrine (NE: 50 μM), Dopamine (DOPA: 40 μM) and Neurotrophin-3 (NT3: 50 ng/ml). After that, 20 μl of aseptically prepared 5 mg/mL MTT was added, adding it also to an additional quadruplicate of wells without cells, as an internal control. The cells were again incubated at 37° C. this time for 2 h, allowing the formation of formazan crystals by the mitochondrial dehydrogenase of viable cells. Afterwards, the media was carefully removed, and the formazan crystals were solubilised by adding 200 μl of MTT solvent per well, a solution that contains acidified isopropanol (4 mM HCl and 0.1% Nondet P-40 in isopropanol). The crystals were further dissolved by gently pipetting the solution up and down being the resulting purple solution spectrophotometrically measured at 570 nm. An increased absorbance can therefore be directly correlated to the cell number, allowing one to determine the cellular viability after a specific treatment. Since the method is dependent on the physiological state of the cells, the assay was always performed after checking the cellular morphology prior treatment. Moreover, two controls were performed: (1) untreated cells, an internal control to verify the drug-dependent effect; and (2) KCl 200 mM treated cells, in which a cytotoxic concentration was used, thus allowing us to verify the assay fidelity.

Results

FIG. 3 shows histograms in which the response to KCl, BDNF, Glutammate, (S)-3,5-Dihydroxyphenylglycine (DHPG), AMPA, NMDA, Acetylcholine, Norepinephrine, Dopamine and 5-HT, measured in RLU for each splice variant (5′UTR) is shown. Data are expressed as ratio F-luc/R-Luc (triplicate experiments). Error bars=SE. (ANOVA on ranks *=p<0.05). These results show that the different agonists are able to modulate the reporter gene expression in a specific and transcript-dependent way (FIG. 3). In particular KCl, BDNF, Glutamate and Norepinephrine stimulation are able to significantly increase the translation of exon1, 2B and 4-containing transcripts; the expression of exon 3-containing plasmid is only induced by the general KCl stimulation, instead exon 8 seem to be responsive to both BDNF and Glutamate; but only the latter is able to induce the expression of exon 2C containing plasmid. In order to further analyze Glutamate stimulation, the activation of the specific glutamate ionotropic receptors (AMPA and NMDA) and the metabotrophic receptor (DHPG) was investigated: results show that the enhancement of exon2C-containing expression is mediated by NMDA receptor but translation of the other BDNF transcripts is not stimulated by these specific agonists, suggesting the need of multiple glutamate receptor stimulation in order to increase the reporter expression. Norepinephrine specific increased exon2C and exon7 reporter expression. Among the agonists that were assayed in this experiment, Serotonin was the agonist which enhanced the largest number of BDNF splice variants as it resulted able to increase the translation of every BDNF exons-containing plasmid, with the exception of those containing exon 2A, 4 and 8.

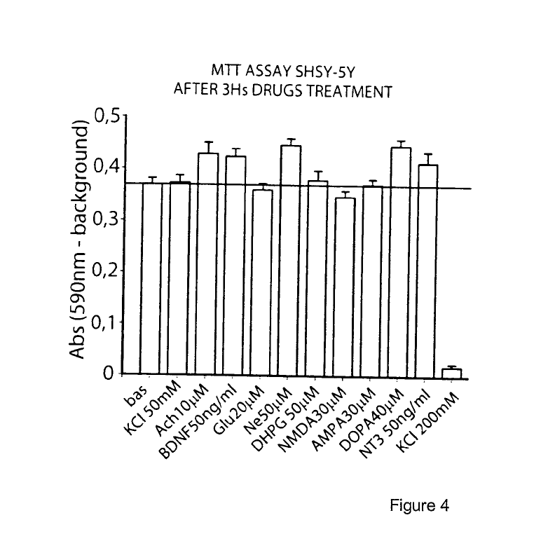

None of the drugs used at the indicated concentrations caused death in SY-SY5Y human neuroblastoma cells (FIG. 4). It was observed that Acetylcholine (ACh 10 μM), Norepineprhine (NE, 50 uM), Dopamine (DOPA, 40 μM) have a neurotrophic effect as they promote a small increase in cell survival similar to that seen with the two neurotrophins BDNF and NT3 (both 50 ng/ml).

### Example 5

Response of BDNF CDS and 3′UTR Variants to Different Agonists

Basal and drug-induced translation of a luciferase reporter gene driven by BDNF short and long 3′UTR or coding region (CDS) sequences were measured. One day prior transfection, undifferentiated neuro-blastoma SH-SY5Y cells were seeded into a 96-well white plate with clear bottom (PBI International) at a confluency of 50-60% and cultured overnight. Plasmids containing the BDNF 3′UTR short or long or CDS were then transiently transfected into SH-SY5Y cells using Lipofectamine™ 2000 (Invitrogen) at high cell density (70-75%), according to the manufacturer's protocol. 24 hours after transfection, the cells were treated with the listed inhibitors at the specified concentrations: KCl (50 mM), Glutammate (GLU: 20 μM), S)-3,5-Dihydroxyphenylglycine (DHPG: 50 μM), AMPA (30 μM), NMDA (30 μM), Acetylcholine (AcH: 30 μM), Norepinephrine (NE: 50 μM), Dopamine (DOPA: 40 μM) and Serotonin (5-HT 50 μM).

BDNF (50 ng/μl) was specifically employed for the stimulation of the 3′UTR long containing plasmid and NT3 (50 ng/μl) for 3′UTR short containing plasmid. Then luciferase assay was performed as previously described according to the manufacturer's instructions (Promega).

Results

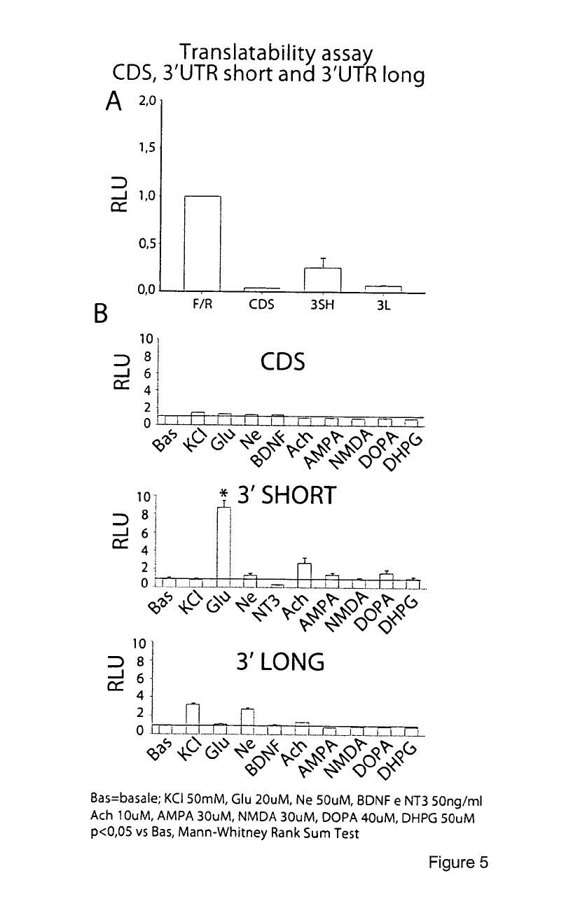

The CDS and the short and long 3′UTR displayed a different profile of responses to drug stimulations as shown in FIG. 5. In particular, BDNF CDS resulted to have no regulatory effect on translation, while the short 3′UTR promoted translation more efficiently than the long 3′UTR (FIG. 5A). In another set of experiments in transfected SHSY-5Y (FIG. 5B), we show that the translation of the CDS and 3′UTR-long transcripts is not enhanced after treatment with the different receptor agonists, even if there is a slightly but not significant increase in 3′UTR-long-containing transcripts translation after the general KCl stimulation. In contrast, there is a striking increase in 3′UTR-short-containing transcripts translation after glutamate treatment. These results confirm the hypothesis that the BDNF CDS is translated at low levels in basal conditions and that it needs the 5′UTR and 3′UTR regions in order to become sensitive to the different stimuli. It is also important to note that the 3′UTR regions are regulated by specific signals and that the short variant is the most responsive one.

### Example 6

Response of BDNF 5′UTR/3′UTR Constructs to Different Antagonists

The present invention can be used not only to identify compounds able to increase BDNF translation, but also to screen for potential inhibitors of BDNF protein production. To this aim a series of vectors were constructed which recapitulate the physiological regulation of BDNF translation in that the firefly luciferase gene was flanked both by 5′UTR regions as well as 3′UTR. These constructs included:

17) pN1-exon1-Fluc-3′UTR short

18) pN1-exon1-Fluc-3′UTR long

19) pN1-exon4-Fluc-3′UTR short

20) pN1-exon4-Fluc-3′UTR long

21) pN1-exon6-Fluc-3′UTR short

22) pN1-exon6-Fluc-3′UTR long.

Inhibitors of 5′UTR regulation are: rapamycin (rap)=mTOR inhibitor (Takei, Inamura et al. 2004); U0126=MEK inhibitor (Kanhema, Dagestad et al. 2006); GF=PKC inhibitor (Heikkila, Jalava et al. 1993).

Inhibitors of 3′UTR regulation: KN62=CaMKII inhibitor (Lu, Hidaka et al. 1996); AuA=Aurora A inhibitor (Soncini, Carpinelli et al. 2006) and PP2=Src tyrosine kinase inhibitor (Perkinton, Sihra et al. 1999).

Aurora kinase A inhibitor (PHA-680632) was a gift from Nerviano Medical Sciences S.r.l. (Milan, Italy); PP2 (1-tert-butyl-3-(4-chlorophenyl)-1H-pyrazolo[3-4]pyrimidin-4-amine), rapamycin was purchased from Ascent Scientific (Bristol, UK), Bisindolylmaleimide I (2-[1-(3-dimethylaminopropyl)-1H-indol-3-yl]-3-(1H-indol-3yl)-maleimide, GF 109203X, Gö 6850), KN62 (1-[N,O-bis-5-isoquinolinesulfonyl)-N-methyl-L-tyrosyl]-4-phenylpiperazine), U0126 (1,4-diamino-2,3-dicyano-1,4-bis(2-aminophenylthio)butadiene), and U0124 (1,4-Diamino-2,3-dicyano-1,4-bis(methylthio)butadiene) from Calbiochem (Damstadt, Germany).

Results

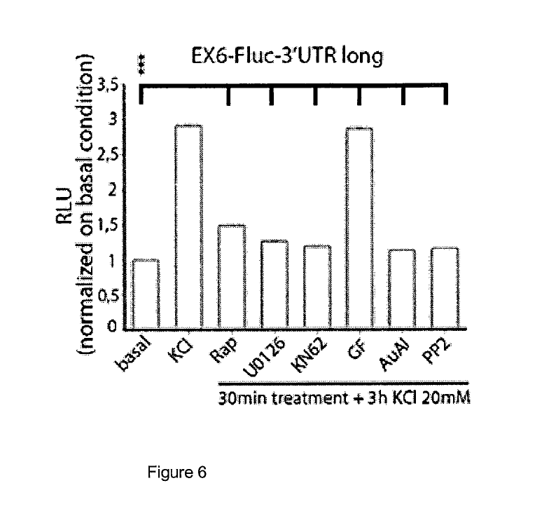

FIG. 6 shows the effects of several inhibitors of the signaling pathways which control KCl-induced protein synthesis of the construct pN1-exon6-Fluc-3′UTR long construct. Relative luciferase units (RLU) describe in absolute values for the Fluc/Rluc (F/R) ratio obtained from each condition—basal, 3 h of 20 mM KCl, 30 min of treatment—rapamycin 20 nM, U0126 50 μM, KN62 20 μM, GF 50 nM, Aurora A inhibitor 10 μM or PP2 20 μM, prior 3 h of depolarization. A value of 1.0 was assumed for the basal condition. The represented data (mean+standard error) corresponds to three independent experiments in quadruplicate ***, P<0.001; **, P<0.01; *, P<0.05, One Way Anova followed by a multiple comparison versus Fluc construct procedure with Holm-Sidak's method.

We show that 30 min of pre-treatment with rapamycin 20 nM, U0126 50 μM, KN62 20 μM, Aurora A inhibitor 10 μM or PP2 20 μM, but not GF 50 nM, completely prevents the three-fold increase in translation observed at the end of 3 h depolarization with 20 mM KCl.

From the above description and the above-noted examples, the advantage attained by the product described and obtained according to the present invention are apparent.

### REFERENCES

Aid, T., A. Kazantseva, et al. (2007). “Mouse and rat BDNF gene structure and expression revisited.” J Neurosci Res 85(3): 525-35.
Aliaga, E. E., I. Mendoza, et al. (2009). “Distinct subcellular localization of BDNF transcripts in cultured hypothalamic neurons and modification by neuronal activation.” J Neural Transm 116(1): 23-32.
Chen, W. G., Q. Chang, et al. (2003). “Derepression of BDNF transcription involves calcium-dependent phosphorylation of MeCP2.” Science 302(5646): 885-9.
Chiaruttini, C., M. Sonego, et al. (2008). “BDNF mRNA splice variants display activity-dependent targeting to distinct hippocampal laminae.” Mol Cell Neurosci 37(1): 11-9.
Garzon, D., G. Yu, et al. (2002). “A new brain-derived neurotrophic factor transcript and decrease in brain-derived neurotrophic factor transcripts 1, 2 and 3 in Alzheimer's disease parietal cortex.” J Neurochem 82(5): 1058-64.
Heikkila, J., A. Jalava, et al. (1993). “The selective protein kinase C inhibitor GF 109203X inhibits phorbol ester-induced morphological and functional differentiation of SH-SY5Y human neuroblastoma cells.” Biochem Biophys Res Commun 197(3): 1185-93.
Huang, E. J. and L. F. Reichardt (2001). “Neurotrophins: roles in neuronal development and function.” Annu Rev Neurosci 24: 677-736.
Jiang, X., J. Zhou, et al. (2009). “Human BDNF isoforms are differentially expressed in cocaine addicts and are sorted to the regulated secretory pathway independent of the Met66 substitution.” Neuromolecular Med 11(1): 1-12.
Kanhema, T., G. Dagestad, et al. (2006). “Dual regulation of translation initiation and peptide chain elongation during BDNF-induced LTP in vivo: evidence for compartment-specific translation control.” J Neurochem 99(5): 1328-37.
Lu, Y. T., H. Hidaka, et al. (1996). “Characterization of a calcium/calmodulin-dependent protein kinase homolog from maize roots showing light-regulated gravitropism.” Planta 199(1): 18-24.
Pattabiraman, P. P., D. Tropea, et al. (2005). “Neuronal activity regulates the developmental expression and subcellular localization of cortical BDNF mRNA isoforms in vivo.” Mol Cell Neurosci 28(3): 556-70.
Perkinton, M. S., T. S. Sihra, et al. (1999). “Ca(2+)-permeable AMPA receptors induce phosphorylation of cAMP response element-binding protein through a phosphatidylinositol 3-kinase-dependent stimulation of the mitogen-activated protein kinase signaling cascade in neurons.” J Neurosci 19(14): 5861-74.
Pruunsild, P., A. Kazantseva, et al. (2007). “Dissecting the human BDNF locus: bidirectional transcription, complex splicing, and multiple promoters.” Genomics 90(3): 397-406.
Roth, T. L., F. D. Lubin, et al. (2009). “Lasting epigenetic influence of early-life adversity on the BDNF gene.” Biol Psychiatry 65(9): 760-9.
Sakata, K., N. H. Woo, et al. (2009). “Critical role of promoter IV-driven BDNF transcription in GABAergic transmission and synaptic plasticity in the prefrontal cortex.” Proc Natl Acad Sci USA 106(14): 5942-7.
Soncini, C., P. Carpinelli, et al. (2006). “PHA-680632, a novel Aurora kinase inhibitor with potent antitumoral activity.” Clin Cancer Res 12(13): 4080-9.
Takei, N., N. Inamura, et al. (2004). “Brain-derived neurotrophic factor induces mammalian target of rapamycin-dependent local activation of translation machinery and protein synthesis in neuronal dendrites.” J Neurosci 24(44): 9760-9.
Tongiorgi, E. and G. Baj (2008). “Functions and mechanisms of BDNF mRNA trafficking.” Novartis Found Symp 289: 136-47; discussion 147-51, 193-5.
Tongiorgi, E. (2008). “Activity-dependent expression of brain-derived neurotrophic factor in dendrites: facts and open questions.” Neurosci Res. 61(4):335-46
Zuccato, C., D. Liber, et al. (2005). “Progressive loss of BDNF in a mouse model of Huntington's disease and rescue by BDNF delivery.” Pharmacol Res 52(2): 133-9.

1. A method of screening for modulators of the translation of brain-derived neurotrophic factor (BDNF) comprising the steps of:
a) transfecting a mammalian cell with a nucleic acid construct, said nucleic acid construct comprising:
a reporter gene; and
a 5′ untranslated (5′UTR) exon of a mammalian BDNF gene;
b) contacting said mammalian cell with a screening compound;
c) detecting the luminescence produced by said mammalian cell of step b).

2. The method according to claim 1, wherein said nucleic acid construct further comprises a 3′ untranslated (3′UTR) exon of a mammalian BDNF.

3. A method of screening for modulators of a brain-derived neurotrophic factor (BDNF) comprising the steps of:
a) transfecting a mammalian cell with a nucleic acid construct, said nucleic acid construct comprising:
a reporter gene; and
a 3′ untranslated (3′UTR) exon of a mammalian BDNF gene;
b) contacting said mammalian cell with a screening compound;
c) detecting the luminescence produced by said mammalian cell of step b).

4. The method according to claim 1, wherein said 5′ untranslated (5′UTR) exon of a mammalian BDNF gene of step a), is the 5′ untranslated (5′UTR) exon of the rat BDNF gene.

5. The method according to claim 4, wherein said 5′ untranslated (5′UTR) exon of the rat BDNF gene is selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 or SEQ ID NO. 10.

6. The method according to anyone of claim 2 or 3, wherein said 3′ untranslated (3′UTR) exon of a mammalian BDNF is the 3′ untranslated (3′UTR) exon of rat BDNF gene.

7. The method according to claim 6, wherein said 3′ untranslated (3′UTR) exon of the rat BDNF gene is selected from the group consisting of SEQ ID NO. 13 and SEQ ID NO. 14.

8. The method according to anyone of claims 1 to 7, wherein said mammalian cell grows in adhesion.

9. The method according to claim 8, wherein said mammalian cell is chosen from the group consisting of SH-SY5Y cells, SK-N-SH cells, SK-N-BE cells, NIH-3T3 cells, HeLa cells, HEK 293 cells, and CHO cells.

10. The method according to anyone of claims 1 to 7, wherein said screening compound is a BDNF-translation agonist.

11. The method according to claim 10, wherein said screening compound is selected from the group consisting of ion-channels agonists, neurotransmitters synthesis, release and re-uptake agonists, neurotransmitter receptors agonists, growth-factors receptors agonists, translation factors agonists and intracellular signaling agonists.

12. The method according to anyone of claims 1 to 7, wherein said screening compound is a BDNF-translation antagonist.

13. The method according to claim 12, wherein said screening compound is selected from the group consisting of ion-channels antagonists, neurotransmitters synthesis, release and re-uptake antagonists, neurotransmitter receptors antagonists, growth-factors receptors antagonists, translation factors antagonists and intracellular signaling antagonists.

14. The method according to anyone of claims 1 to 13, wherein said reporter gene is a luciferase gene.

15. The method according to claim 14, wherein said luciferase gene is chosen from the group consisting of Firefly luciferase and Renilla luciferase.

16. The method according to anyone of claims 1 to 15, wherein said method of screening is carried out by the high throughput screening (HTS) technique.

17. A nucleic acid construct comprising either:
a) a reporter gene; and
b) a 5′ untranslated (5′UTR) exon of the rat BDNF gene selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 or SEQ ID NO. 10;
or
a) a reporter gene; and
b) a 3′ untranslated (3′UTR) exon of the rat BDNF gene selected from the group consisting of SEQ ID NO. 13 and SEQ ID NO. 14.
or
a) a 5′ untranslated (5′UTR) exon of the rat BDNF gene selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 or SEQ ID NO. 10; and
b) a reporter gene; and
c) a 3′ untranslated (3′UTR) exon of the rat BDNF gene selected from the group consisting of SEQ ID NO. 13 and SEQ ID NO. 14.

18. The nucleic acid construct according to claim 17, wherein said reporter gene is a luciferase gene.

19. The nucleic acid construct according to claim 18, wherein said luciferase gene is chosen from the group consisting of Firefly luciferase and Renilla luciferase.

20. A plasmid comprising the nucleic acid construct according to claim 17, 18 or 19.


SEQUENCE LISTING (14 sequences)

SEQ ID NO: 1 - DNA, 613 bp, Rattus norvegicus
taaagcggtagccggctggtgcaggaaagcaacaagttccccagcggtcttcccgccctagcctgacaaggcgaaggttttcttacctggcgacagggaaatctcctgagccgagctcatctttgccacagccccaggtgtgacctgagcagtgggcaaaggagcggcgtgcaaattggattatttgtatgggggtactctgaaactccctcactttttctgggaactttttgtgctagggcgcagtgacaggcgttgagaaagctgcttcaggaaacgcccgctatatagcagggcagttggacagtcattggtaacctcgctcattcattagaatcacgtaagaactcaaagggaaacgtgtctctcagaatgagggcgtttgcgtaaatctataggtttttcaacatcgatgccagttgctttgtcttctgtaatcgccaaggtggatgagagttgaagcttgcggatattgcaaagggttattagattcataagtcacaccaagtggtgggcgatccactgagcaaagccgaacttctcacatgatgacttcaaacaagacacattaccttccagcatctgttggggagacgagattttaagacactga

SEQ ID NO: 2 - DNA, 205 bp, Rattus norvegicus
gctttggcaaagccatccgcacgtgacaaaacgtaaggaagtggaagaaaccgtctagagcaatatcaagtaccacttaattagagaatatttttttaaccttttcctcctgctgcgccgggtgtgtgatccgggcgagcagagtccattcagcaccttggaacagagccagcggatttgtccgaggtggtagtacttcatccag

SEQ ID NO: 3 - DNA, 415 bp, Rattus norvegicus
gctttggcaaagccatccgcacgtgacaaaacgtaaggaagtggaagaaaccgtctagagcaatatcaagtaccacttaattagagaatatttttttaaccttttcctcctgctgcgccgggtgtgtgatccgggcgagcagagtccattcagcaccttggaacagagccagcggatttgtccgaggtggtagtacttcatccaggtattcttttcctcgctgtcaagccaacccggtgtcgcccttaaaaagcgtcttttccgaggttcggctcacactgagatcggggctggagagagagtcagattttggagcggagcgtttggagagccagccccagtttggtcccctcattgagctcgctgaagttggcttcctagcggtgtaggctggaatagactcttggcaagctcc

SEQ ID NO: 4 - DNA, 500 bp, Rattus norvegicus
gctttggcaaagccatccgcacgtgacaaaacgtaaggaagtggaagaaaccgtctagagcaatatcaagtaccacttaattagagaatatttttttaaccttttcctcctgctgcgccgggtgtgtgatccgggcgagcagagtccattcagcaccttggaacagagccagcggatttgtccgaggtggtagtacttcatccaggtattcttttcctcgctgtcaagccaacccggtgtcgcccttaaaaagcgtcttttccgaggttcggctcacactgagatcggggctggagagagagtcagattttggagcggagcgtttggagagccagccccagtttggtcccctcattgagctcgctgaagttggcttcctagcggtgtaggctggaatagactcttggcaagctccgggttggtatactgggttaactttgggaaatgcaagtgtttatctccaggatctagccaccggggtggtgtaagccgcaaagaag

SEQ ID NO: 5 - DNA, 215 bp, Rattus norvegicus
gccctcacgattctcgctggatagttctttatgttttgggtgatttttgttttttgtttgtttggttttttttttttttaatttttacccctttctattttccctccccgagagttccgggctctggcttggagggctcctgctttctcaagggaaggggagccgctgagactgcgctccactccctgccgggctggatgcttcattgagcccag

SEQ ID NO: 6 - DNA, 335 bp, Rattus norvegicus
acccactttcccattcaccgaggagaggactgctcgcgctgccgctccccccacccacccccggcgagctagcatgaaatctcccagtctctgcctagatcaaatggagcttctcactgaaggcgtgcgagtattacctccgccatgcaatttccactatcaataatttaacttctttgctgaagaacaggagtacatatcggccaccaaagactcgcccccctcccccttttaactgaagagaaggggaaatatatagtaagagtctagaaccttggggaccggtcttccccagagcagctgccttgatgtttactttgacaagtagtgactga

SEQ ID NO: 7 - DNA, 81 bp, Rattus norvegicus
aaaccataaccccgcacactctgtgtagtttcattgtgtgttcgctttttctagctctgtggtgcgggaaggtgcgggaag

SEQ ID NO: 8 - DNA, 348 bp, Rattus norvegicus
ccaatcgaagctcaaccgaagagctaaataatgtctgaccccagtgcctggcgctggctgagctctgggtgcccgccgctgccgccgcgccggggcgcacccgctggctggctgtcgcacggtccccattgcgcccgggactccccggcttggagaaggaaaccgcctggggcggcgcgccacctccgcctggcaggctttgatgagaccgggttccctcagctcgccaccgctgctttggggcagacgagaaagcgcacggggcccagggcagggcgcagggaccaggagcgtgacaacaatgtgactccactgccggggatccgagagctttgtgtggaccctgag

SEQ ID NO: 9 - DNA, 213 bp, Rattus norvegicus
cactgtcacctgctttctagggagtattaccaaagcttcttacaagtccaaggtcaacataactaggaacttgggatcattcttgtcactgggacctgaaagggtctgcggaactccaggtcccacagcttgtatccgaccctctctgtccatccagcgcacctctttaggcatcctccagaggaagtgaaagttttgactttcatccgggag

SEQ ID NO: 10 - DNA, 256 bp, Rattus norvegicus
gttatagagttggatgcaagcgtaacccgaaggacaactggatgtgtggaagcaccagttttctccatgtgctcaggctaatcctcgttaatactcagaacgttgagtttctacggaatgggttgcatgtccatacatgttttggtatctacccacacactttcatgtggtatgactgtgcatcccaggagaaaggctgtggtgtgtgtctctgcgcctcagtggagctgaacaaacgattgctgaaaatggtgtc

SEQ ID NO: 11 - DNA, 1653 bp, Rattus norvegicus
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 12 - DNA, 940 bp, Rattus norvegicus
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaacgggccc

SEQ ID NO: 13 - DNA, 320 bp, Rattus norvegicus
tggatttatgttgtatagattatattgagacaaaaattatctatttgtatatatacataacagggtaaattattcagttaagaaaaagataattttatgaactgcatgtataaatgaagtttatacagtacagtggttctacaatctatttattggacatatccatgaccagaaagaaacagtcatttgcgcacaactttaaaagtctgcattacattcctcgataatgttgtggtttgttgccgttgccaagaattgaaaacaaaaagttaaaaaaaataataaattgcatgctgctttaattgtgaattgataataaa

SEQ ID NO: 14 - DNA, 2791 bp, Rattus norvegicus
tggatttatgttgtatagattatattgagacaaaaattatctatttgtatatatacataacagggtaaattattcagttaagaaaaagataattttatgaactgcatgtataaatgaagtttatacagtacagtggttctacaatctatttattggacatatccatgaccagaaagaaacagtcatttgcgcacaactttaaaagtctgcattacattcctcgataatgttgtggtttgttgccgttgccaagaattgaaaacaaaaagttaaaaaaaataataaattgcatgctgctttaattgtgaattgataataaactgtccctctttcagaaaacagacaaaaaaacaaaaaacaaaaaaaagcaaaaacaaaaatttgaaccaaaacattccgtttacattttagacactaagtatcttcgttcttgttagtactctgttctactgctttcaacttctcatagcgttggaattaaaactatgtcaaggtgctgttgtcattgctttactggcttaggggatggggaacgggaggggtagatttctgtttgttttgtgttttatttcgtttgtttgtttgttttgtttttttagttccacccggagtagggatggagaaaatttcttcactctccattctggttgataaagcgttacatttgtatgttgtaaaaaatgtttgcaaaatccaatcagatgactggaaaacgaataaaaattaaggcaactgaataaaatgctcacacaacactgcccatgatgtatctccctggtcccccaggtcactcttctggcatgggtcagggaaagctgcttttattggaaagaccagcatttgtttaaagcacattctttccctccctcctcccattttggtccctttcttttttgttttgttttaagaaagaaaattaagttgcgcgctttgaaatattttatcactgctgtgaacagatgaacaatgtgtgtcatttcatgacactcgtggaaaacagtgattttttttttattttttgccctaaggagaaacaagtaagaataaccgaaaatgttctttttttttaaaggcataaacagtggataagttataatatggcctaacaatgtttgcagataaaagatattgcatacagccagatactagagcagggatccacactgccactgaaatgcgactgaatggccctgtggaggctaagtggagctgacatactatttcctggcagagcaggaggaatttctgagtggccatcctgaggtctagatggaggtggggaatggtacttgagacattcctaaaggaaggctcggaagcacccttcagagcaggctctggaatgatgtgtcaagtttcttaggccttctgctttaagtgcctacgttacctaacagtgctcaagaggttctcaattggagaaccacactcaaatccatttatggcctccatcccattttaaataattatggataaagttggattaacctggagcagctttggatccaaatatggcatagcagtgatgctatcagtgcagcatgatgggaaatgtttgctgtgaagagacttaactttctttgcgcttagacttcaggaagcctaggttttatttatttatttttttgagacattttggtgaaaggaaaaaagaaagaagaaaacaaacaaacaaaaccagaaaaagcaccaaaacttaggcagaatgagcaatgtctgtctgtaagggctagaatgacaaggcataggaaggtgctttcactgtgaaagagacaagaacacaggaggaaatactgcttaagtgaagagcacagaaagctcctgatagttctgtccattcagcacaagggtcccttctacactttacctcttggggttaggagaagtcaagctggaagcctgaatgaatggccccaatgagaactagtgttaagcccatttccctagtgaggttttccgccagcgcgaatgtgttagtggttacctgactgggctcctgggcatcagaaaaagaggcaaacaattgcttcatcttaggagtggaaagggtgaaacaaagtggctgtccactgtgactcagggagtgaagataccatcagcaaatagtttctttttgttcaactgttcctttagaactagtctgtcttctggagtcccactgaatcccctgtttttggaagacttcacgtagcctagattgttttgtgccgtttgacaacattaatctctgtcatcatttttaacctattaaggaatgctttgaatatctgctatatgctaactttttgcagcttcattctgagagacgttagtcaaacaaataaaaggagccccatcacaatctcacggtattcgaagggccaggtcgattaggtggcttcataggagaccctccgcaactgtgtggtcagtggctggctctcatacccactaagatacatcatagctccatgtcggtggtttatgttgacctgagattgatttgttaaaatctctcctctgtttctgttcgttctgtttccgtcctgttctgttctgttctgttctgaaagtcttgctgtggtctctttttggcagaagtgtttcatgcatggcagcaggcctgatgctttttatagtgattcccattgaaactgtaagtaaatgtctgtggccttgttctctctatggtaaagatattattcaccatgtaaaacaagaaaaatatttattgtattttagtatatttatataattatgttattgaaaaaaattggcattaaaacttaaccacatcagaagcctattgtaa